(19) United States
(12) Patent Application Publication
(10) Pub. No.: US 2022/0104781 A1
(43) Pub. Date: Apr. 7, 2022

(54) NUCLEAR MEDICINE DIAGNOSTIC APPARATUS

(71) Applicant: CANON MEDICAL SYSTEMS CORPORATION, Tochigi (JP)

(72) Inventor: Manabu TESHIGAWARA, Otawara (JP)

(73) Assignee: CANON MEDICAL SYSTEMS CORPORATION, Tochigi (JP)

(21) Appl. No.: 17/495,967

(22) Filed: Oct. 7, 2021

(30) Foreign Application Priority Data
Oct. 7, 2020 (JP) 2020-169599

(51) Int. Cl. A61B 6/03; G01T 1/20; G01T 1/202

(52) U.S. Cl. CPC A61B 6/037; G01T 1/202; G01T 1/2018

(57) ABSTRACT

A nuclear medicine diagnostic apparatus according to an embodiment includes a positron emission tomography (PET) detector, a scatterer, and processing circuitry. The scatterer is provided inside the PET detector. The processing circuitry detects a gamma ray scattered by the scatterer with the PET detector to identify a single event.

### CROSS-REFERENCE TO RELATED APPLICATIONS

This application is based upon and claims the benefit of priority from Japanese Patent Application No. 2020-169599, filed on Oct. 7, 2020; the entire contents of which are incorporated herein by reference.

### FIELD

Embodiments described herein relate generally to a nuclear medicine diagnostic apparatus.

### BACKGROUND

Compton cameras are known as gamma ray detectors. A Compton camera includes a scatterer and an absorber so that gamma rays incident on the scatterer cause Compton scattering with electrons in the scatterer and change their directions and the scattered gamma rays with the changed directions are detected by the absorber. A Compton scattering point is specified in the scatterer, and the energy and the detection position of a scattered gamma ray are measured in the absorber.

The energy given to the electron by the gamma ray due to scattering, i.e., the energy lost from the incident gamma ray, and the scattering angle have the relation described by the Klein-Nishina formula. The Compton camera uses this relation to specify the incident direction of the gamma ray in the shape of a cone with the Compton scattering point as an apex. Specifically, the Compton camera uses the straight line connecting the Compton scattering point in the scatterer and the absorption point of the scattered gamma ray in the absorber as an axis, uses the Compton scattering point as an apex and forms, for a plurality of incident gamma rays, a cone that is formed with the scattering angle based on the energy (the energy of the recoil electron) given to the electron by the gamma ray. In the Compton camera, a large number of such cones are superimposed to depict the image of a gamma-ray source.

In recent years, there have been known electron-track detection Compton cameras having the function of detecting the track of a recoil electron by Compton scattering in the scatterer, applying the law of conservation of momentum to the vicinity of scattering, and limiting the arrival direction of an incident gamma ray to a part of directions on the cone. That is, there have been known Compton cameras that may handle gamma rays like visible light with geometric optics.

### BRIEF DESCRIPTION OF THE DRAWINGS

FIG. 1 is a diagram illustrating an example of a configuration of a PET apparatus according to a first embodiment;

FIG. 2 is a diagram illustrating processing by an identification function according to the first embodiment;

FIG. 3 is a diagram illustrating an example of generation of single event counting information by the identification function according to the first embodiment;

FIG. 4 is a diagram illustrating an example of the generation of the single event counting information by the identification function according to the first embodiment;

FIG. 5 is a diagram illustrating an example of an LOR according to the first embodiment;

FIG. 6A is a diagram illustrating an installation example of a scatterer detector according to the first embodiment;

FIG. 6B is a diagram illustrating an installation example of the scatterer detector according to the first embodiment;

FIG. 7 is a flowchart illustrating a processing procedure by the PET apparatus according to the first embodiment; and

FIG. 8 is a diagram illustrating an example of a configuration of a SPECT apparatus according to a second embodiment.

### DETAILED DESCRIPTION

A nuclear medicine diagnostic apparatus according to an embodiment includes a positron emission tomography (PET) detector, a scatterer, and processing circuitry. The scatterer is provided inside the PET detector. The processing circuitry detects a gamma ray scattered by the scatterer with the PET detector to identify a single event.

Embodiments of the nuclear medicine diagnostic apparatus according to the present application are described below in detail with reference to the attached drawings. The nuclear medicine diagnostic apparatus according to the present application is not limited to the embodiments described below. Furthermore, the embodiment may be combined with other embodiments or conventional techniques as long as there is no contradiction in processing details.

### First Embodiment

A nuclear medicine diagnostic apparatus according to a first embodiment is described below. In the first embodiment, a positron emission tomography (PET) apparatus is described as an example of a nuclear medicine diagnostic apparatus.

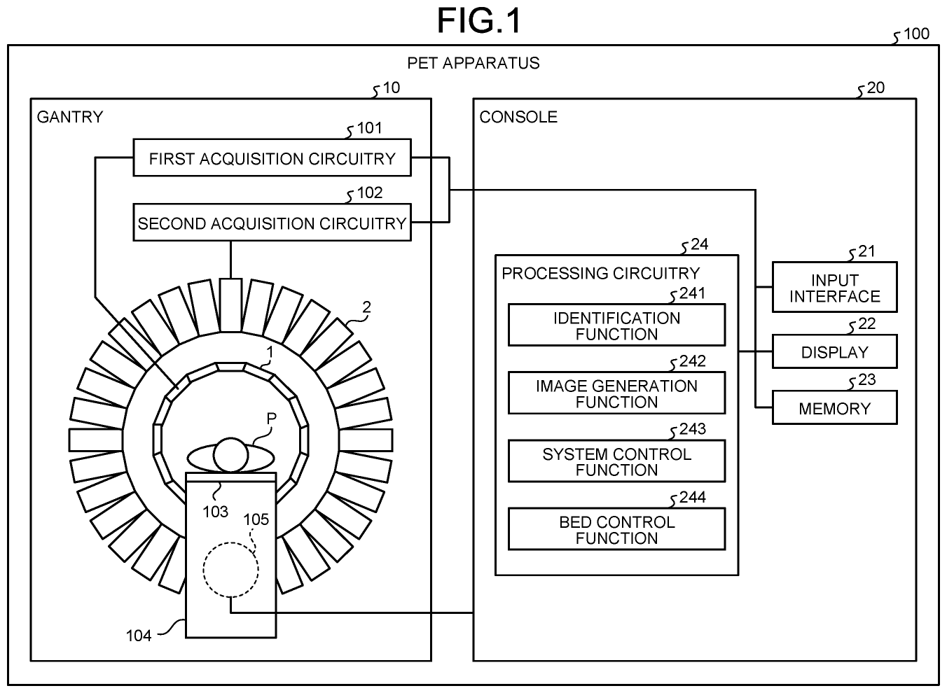

FIG. 1 is a diagram illustrating an example of a configuration of a PET apparatus 100 according to the first embodiment. As illustrated in FIG. 1, the PET apparatus 100 according to the first embodiment includes a gantry 10 and a console 20.

The gantry 10 includes a scatterer detector 1, a PET detector 2, first acquisition circuitry 101, second acquisition circuitry 102, a top plate 103, a bed 104, and a bed drive unit 105 to detect gamma rays emitted from a living tissue that takes up a positron-emitting radionuclide administered to a subject P and thus generate counting information for reconstructing a PET image.

The scatterer detector 1 causes Compton scattering of an annihilation gamma ray emitted from the subject P and detects a Compton scattering point. For example, as illustrated in FIG. 1, the scatterer detector 1 is provided inside the PET detector 2 in a circular shape. Specifically, the scatterer detector 1 is provided so as to annularly surround the subject P. The scatterer detector 1 is an example of the scatterer.

For example, the scatterer detector 1 includes a scatterer including a plurality of pixels to cause Compton scattering of annihilation gamma rays incident on the respective pixels. The scatterer detector 1 detects the recoil electron generated by Compton scattering for each pixel and thus detects the Compton scattering point. Any material such as Si may be used as a scatterer. The scatterer detector 1 may be configured to measure the energy of a recoil electron.

The scatterer detector 1 may also be configured to detect the track of a recoil electron. In such a case, for example, the scatterer detector 1 includes a time projection chamber (TPC). For example, the TPC has the inside filled with a gas, which is a scatterer, and includes a collector that amplifies and detects ionization electrons. The TPC has a formed drift region that is substantially perpendicular to the collector and has a uniform electric field acting therein.

The TPC causes Compton scattering of the incident annihilation gamma ray due to the interaction with the gas (electron in the gas molecule) in the chamber. As a result, the recoil electron generated by Compton scattering passes while continuously ionizing gas molecules to generate an electron cloud including a large number of ionization electrons on the track. The generated electron cloud drifts to the collector in the drift region while retaining substantially the same shape as that of the track of the recoil electron due to the force applied from the electric field. The TPC detects an arrival position (X, Y coordinates) of the electron cloud at the collector. The TPC detects a distance (Z coordinate) from a collecting unit to the track based on the difference between the time when Compton scattering occurs and the time when the collector detects the ionization electron and the drift velocity of the ionization electron. Thus, the TPC acquires the three-dimensional position of the track of the recoil electron.

The PET detector 2 detects a gamma ray by detecting a scintillation light (fluorescence), which is a light re-emitted when a substance in an excited state due to the interaction between a scintillator and an annihilation gamma ray emitted from a positron in the subject P transitions to a ground state again. Simultaneously, the PET detector 2 detects energy information on the annihilation gamma ray emitted from the positron in the subject P.

Here, the PET detector 2 detects a scattered gamma ray generated by Compton scattering in the scatterer detector 1. Specifically, the PET detector 2 detects the incident position and the energy of the scattered gamma ray.

As illustrated in FIG. 1, the PET detector 2 is provided outside the scatterer detector 1 so as to annularly surround the subject P. For example, the PET detector 2 is a detector of a photon counting method, anger type, or non-anger type. An anger type detector includes a scintillator, a photodetection element, and a light guide. A non-anger type includes a scintillator (scintillator piece) and a photodetection element but not a light guide, and the scintillator piece and the photodetection element are optically bonded one-on-one.

The scintillator converts the incident annihilation gamma rays emitted from positrons in the subject P into scintillation lights (scintillation photons, optical photons) and outputs the scintillation lights. The scintillator is formed of scintillator crystals suitable for energy measurement, such as LaBr3 (lanthanum bromide), LYSO (lutetium yttrium oxyorthosilicate), LSO (lutetium oxyorthosilicate), LGSO (lutetium gadolinium oxyorthosilicate) or BGO, GAGG (gadolinium aluminum gallium garnet), or LuAG (lutetium aluminum garnet), and is arranged in for example two dimensions.

For example, SiPM (silicon photomultiplier) or a photomultiplier tube is used as a photodetection element. The photomultiplier tube includes a photocathode that receives a scintillation light and generates a photoelectron, a multi-stage dynode that gives an electric field to accelerate the generated photoelectron, and an anode that is an electron outflow port to multiply the scintillation light output from the scintillator and convert it into an electrical signal.

The light guide is formed of a plastic material, or the like, having desired light transmission to transmit the scintillation light output from the scintillator to the photodetection element such as SiPM or photomultiplier tube.

The PET detector 2 may be a direct conversion detector using a semiconductor such as CZT (cadmium zinc telluride) as well as the indirect conversion detector using the scintillator described above.

The first acquisition circuitry 101 generates first counting information from an output signal of the scatterer detector 1 and stores the generated first counting information in a memory 23 of the console 20.

The first acquisition circuitry 101 converts the output signal of the scatterer detector 1 into digital data to generate the first counting information. The first counting information includes the detection position and the detection time of the Compton scattering point. For example, the first acquisition circuitry 101 specifies the pixel that has detected the recoil electron generated by Compton scattering. The first acquisition circuitry 101 specifies the pixel number for identifying the position of the specified pixel.

The first acquisition circuitry 101 specifies the detection time when the scatterer detector 1 detects the recoil electron generated by Compton scattering. The detection time may be the absolute time or an elapsed time from the scan start time. As described above, the first acquisition circuitry 101 generates the first counting information including the pixel number and the detection time.

When the scatterer detector 1 is a TPC, the first acquisition circuitry 101 generates the first counting information including the position of the Compton scattering point, the detection time, and the information on the track of the recoil electron based on the output signal of the TPC and stores the generated first counting information in the memory 23. For example, the first acquisition circuitry 101 generates the first counting information including the information on the three-dimensional position of the track of the recoil electron and the detection time.

The second acquisition circuitry 102 generates second counting information from the output signal of the PET detector 2 and stores the generated second counting information in the memory 23 of the console 20.

The second acquisition circuitry 102 converts the output signal of the PET detector 2 into digital data to generate the second counting information. The second counting information includes the detection position of the gamma ray, the energy value, and the detection time. For example, the second acquisition circuitry 102 specifies a plurality of photodetection elements that have converted scintillation lights into electrical signals at the identical timing. The second acquisition circuitry 102 specifies the scintillator number indicating the position of the scintillator on which the gamma ray is incident. A specifying unit may conduct centroid computation based on the position of each photodetection element and the intensity of the electric signal to specify the position of the scintillator on which the gamma ray is incident. When the element sizes of the scintillator and the photodetection element correspond to each other, the scintillator corresponding to the photodetection element from which the output is obtained may be specified as the position of the scintillator on which the gamma ray is incident.

The second acquisition circuitry 102 executes integral calculation on the intensity of the electric signal output from each photodetection element to specify the energy value of the gamma ray incident on the PET detector 2. The second acquisition circuitry 102 may specify the energy value by ToT (time over threshold) measurement as well as the above-described integral calculation. In such a case, for example, the second acquisition circuitry 102 measures the time (ToT) during which the electric signal output from each photodetection element exceeds a set threshold and executes non-linear correction on the measurement result to specify the energy value. The second acquisition circuitry 102 specifies the detection time when the PET detector 2 detects the scintillation light due to the gamma ray. The detection time may be the absolute time or an elapsed time from the imaging start time. In this manner, the second acquisition circuitry 102 generates the second counting information including the scintillator number, the energy value, and the detection time.

Here, the second acquisition circuitry 102 generates the second counting information for the scattered gamma ray that is incident on the PET detector 2 after Compton scattering of the annihilation gamma ray, emitted from the subject P, in the scatterer detector 1 and for the gamma ray that is incident on the PET detector 2 without Compton scattering of the annihilation gamma ray. Specifically, the second acquisition circuitry 102 generates the second counting information including the detection position, the energy value, and the detection time of the scattered gamma ray and the detection position, the energy value, and the detection time of the gamma ray incident on the PET detector without Compton scattering.

The first acquisition circuitry 101 and the second acquisition circuitry 102 are implemented by, for example, a CPU (central processing unit), a GPU (graphical processing unit), or a circuit such as an application specific integrated circuit (ASIC), a programmable logic device (e.g., simple programmable logic device (SPLD), complex programmable logic device (CPLD), and a field programmable gate array (FPGA)).

The top plate 103 is a bed on which the subject P is placed and is provided on the bed 104. The bed drive unit 105 moves the top plate 103 under the control of a bed control function 244 of processing circuitry 24. For example, the bed drive unit 105 moves the top plate 103 to move the subject P into an imaging port of the gantry 10.

The console 20 receives an operation on the PET apparatus 100 by an operator, controls capturing of a PET image, and reconstructs the PET image using the counting information collected by the gantry 10. As illustrated in FIG. 1, the console 20 includes an input interface 21, a display 22, the memory 23, and the processing circuitry 24. The units included in the console 20 are coupled to each other via a bus.

The input interface 21 is implemented by, for making various settings, and the like, a trackball, a switch button, a mouse, a keyboard, a touch pad for performing an input operation by touching an operation surface, a touch monitor having a display screen and a touch pad integrated therein, a non-contact input circuit using an optical sensor, a voice input circuit, etc. The input interface 21 is coupled to the processing circuitry 24 to convert the input operation received from the operator into an electric signal and output the electric signal to the processing circuitry 24. In this description, the input interface 21 is not limited to the one including a physical operating part such as a mouse and a keyboard. Examples of the input interface include an electric signal processing circuit that receives an electric signal corresponding to an input operation from an external input device, which is provided separately from the apparatus, and outputs the electric signal to the processing circuitry 24.

The display 22 is coupled to the processing circuitry 24 to display various types of information and various types of image data output from the processing circuitry 24. For example, the display 22 is implemented by a liquid crystal display, a CRT (cathode ray tube) display, an organic EL display, a plasma display, or a touch panel. According to the present embodiment, for example, the display 22 presents a PET image or presents a GUI (graphical user interface), or the like, for receiving various instructions and various settings from the operator.

The memory 23 stores various types of data used in the PET apparatus 100. The memory 23 is implemented by, for example, a semiconductor memory device such as a RAM (random access memory) or a flash memory, a hard disk, or an optical disk. The memory 23 stores the first counting information acquired by the first acquisition circuitry 101, the second counting information acquired by the second acquisition circuitry 102, single event counting information in which the identification number for identifying the single event is associated with the first counting information and the second counting information, coincidence counting information in which the identification number for identifying the coincidence counting event is associated with a combination of second counting information, the reconstructed PET image, etc.

As illustrated in FIG. 1, the processing circuitry 24 executes an identification function 241, an image generation function 242, a system control function 243, and the bed control function 244. Here, according to the embodiment, each of the processing functions performed by the identification function 241, the image generation function 242, the system control function 243, and the bed control function 244 is stored in the memory 23 in the form of a program executable by a computer. The processing circuitry 24 is a processor that reads and executes a program from the memory 23 to perform the function corresponding to each program. In other words, the processing circuitry 24 having read each program has each function illustrated in the processing circuitry 24 of FIG. 1. In the description of FIG. 1, it is assumed that the single processing circuitry 24 performs the identification function 241, the image generation function 242, the system control function 243, and the bed control function 244, but a plurality of independent processors may be combined to form the processing circuitry 24, and each of the processors may execute a program to perform the function. In other words, each of the above-described functions may be configured as a program, and the single processing circuitry 24 may execute each program. As another example, a specific function may be installed in a dedicated and independent program execution circuit. The processing circuitry 24 is an example of processing circuitry.

The identification function 241 detects the gamma ray scattered by the scatterer with the PET detector 2 to identify a single event. Specifically, the identification function 241 generates the single event counting information based on the first counting information and the second counting information and stores the generated single event counting information in the memory 23. The identification function 241 generates the coincidence counting information based on the second counting information and stores the generated coincidence counting information in the memory 23. The processing of the identification function 241 is described below in detail.

The image generation function 242 reconstructs the PET image using the counting information. Specifically, the image generation function 242 reads the single event counting information stored in the memory 23 and reconstructs the PET image using the read single event counting information. The image generation function 242 reads the coincidence counting information stored in the memory 23 and reconstructs the PET image using the read coincidence counting information. The image generation function 242 reads the single event counting information and the coincidence counting information stored in the memory 23 and reconstructs the PET image using the read coincidence counting information. The image generation function 242 stores the reconstructed PET image in the memory 23.

The system control function 243 controls the gantry 10 and the console 20 to perform the overall control on the PET apparatus 100. For example, the system control function 243 controls imaging in the PET apparatus 100.

The bed control function 244 controls the bed drive unit 105 to control the position of the top plate 103.

The configuration of the PET apparatus 100 according to the first embodiment has been described above. With this configuration, the PET apparatus 100 makes it possible to easily implement a Compton camera in the nuclear medicine diagnostic apparatus. As described above, the Compton camera includes the scatterer and the absorber to specify the incident direction of the gamma ray based on the Compton scattering point in the scatterer, the energy lost from the gamma ray due to Compton scattering, and the detection position and the energy of the scattered gamma ray incident on the absorber and depicts the image of the gamma-ray source.

However, although the incident direction of the gamma ray in the PET apparatus 100 is specified by identifying the line of response by coincidence counting of annihilation gamma rays, limiting the incident direction by coincidence counting is accompanied by a noise called accidental coincidence counting. Therefore, it is expected to implement a clinical Compton camera applicable to medical diagnosis.

In order to implement a Compton camera that may obtain a sufficient image quality applicable to medical diagnosis, it is desirable to configure a detector system with a geometrical arrangement that surrounds the subject. However, in the case of the design of an independent clinical Compton camera, the cost is higher than that of a normal PET apparatus as there is the scatterer in addition to the absorber that corresponds to the PET detector. Furthermore, the independent clinical Compton camera needs to be installed separately from the PET apparatus that has been operated with the already established examination method as medical treatment. Therefore, for designing the independent clinical Compton camera, there are barriers in terms of both cost and operation.

Therefore, in the PET apparatus 100 according to the present embodiment, the Compton camera is implemented by using the existing PET detector as an absorber and simply adding the scatterer. That is, in the PET apparatus 100 according to the present embodiment, by implementing the Compton camera in the PET apparatus, it is possible to specify the incident direction of the gamma ray with the Compton camera as well as coincidence counting of annihilation gamma rays.

The details of processing by the identification function 241 are described below. The identification function 241 identifies a single event based on the detection of the scattered gamma ray by Compton scattering with the scatterer detector 1.

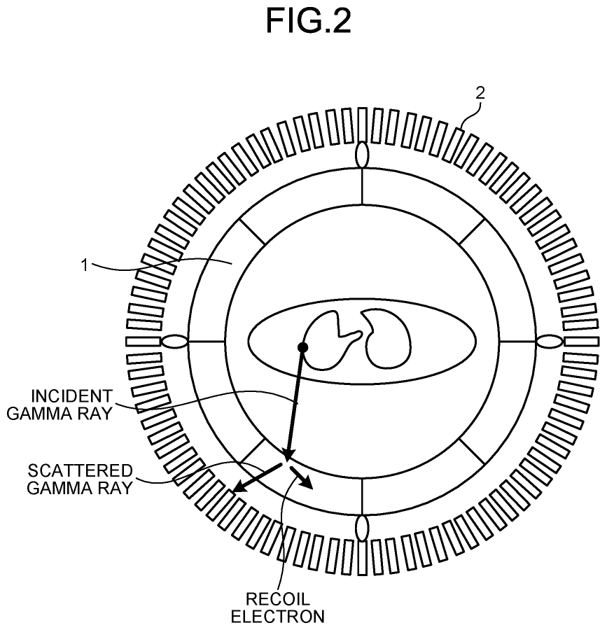

FIG. 2 is a diagram illustrating processing by the identification function 241 according to the first embodiment. As illustrated in FIG. 2, in the PET apparatus 100, the annihilation gamma ray emitted from the subject is incident on the scatterer detector 1 as an incident gamma ray. Although FIG. 2 illustrates only one of the annihilation gamma rays, the annihilation gamma ray is actually emitted in substantially the opposite direction. When the incident gamma ray is incident, Compton scattering occurs in the scatterer detector 1, and a scattered gamma ray and a recoil electron are generated.

The scatterer detector 1 detects the Compton scattering point based on the recoil electron. The first acquisition circuitry 101 generates the first counting information including the position of the Compton scattering point and the detection time based on the output signal from the scatterer detector 1 and stores the first counting information in the memory 23. The first acquisition circuitry 101 sequentially generates the first counting information based on the output signal from the scatterer detector 1 and stores the first counting information in the memory 23.

As illustrated in FIG. 2, the scattered gamma ray generated by the scatterer detector 1 is incident on the PET detector 2. The PET detector 2 detects the incident position of the scattered gamma ray and the energy of the scattered gamma ray. The second acquisition circuitry 102 generates the second counting information including the incident position of the scattered gamma ray, the energy of the scattered gamma ray, and the detection time based on the output signal from the PET detector 2 and stores the second counting information in the memory 23. The second acquisition circuitry 102 sequentially generates the second counting information based on the output signal from the PET detector 2 and stores the second counting information in the memory 23.

The identification function 241 reads the first counting information and the second counting information from the memory 23 to generate the single event counting information having the first counting information and the second counting information associated with each other based on the position of the Compton scattering point, the incident position of the scattered gamma ray, and each detection time. That is, the identification function 241 generates the single event counting information having the Compton scattering point, the incident position of the scattered gamma ray, and the energy of the scattered gamma ray associated with each other.

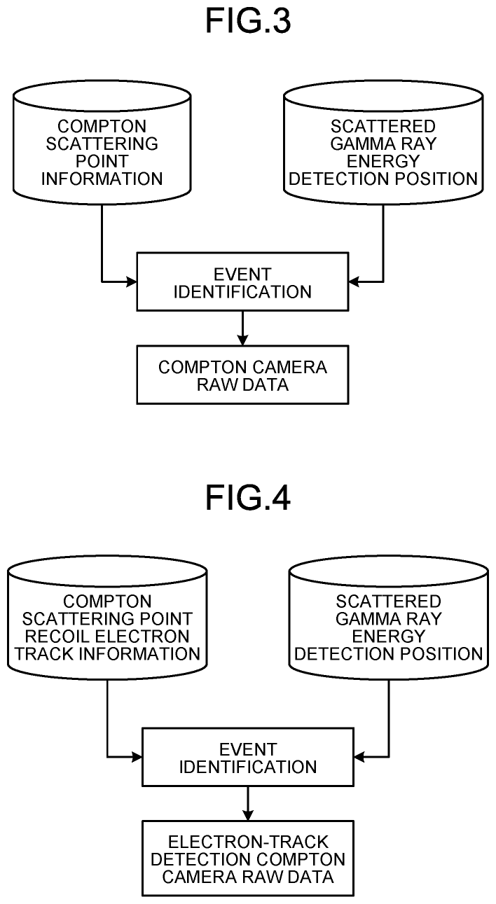

FIG. 3 is a diagram illustrating an example of generation of the single event counting information by the identification function 241 according to the first embodiment. For example, the identification function 241 uses the position of the Compton scattering point, the detection position of the scattered gamma ray, and each detection time to associate the information on the Compton scattering point included in the first counting information with the energy and the detection position of the scattered gamma ray included in the second counting information, as illustrated in FIG. 3, so as to identify the single event. The identification function 241 stores, in the memory 23, the single event counting information including the information on the Compton scattering point included in the first counting information and the energy and the detection position of the scattered gamma ray included in the second counting information.

The identification function 241 uses the first counting information generated by the first acquisition circuitry 101 and the second counting information generated by the second acquisition circuitry 102 to sequentially generate the single event counting information with regard to the annihilation gamma ray emitted from the subject P and stores the single event counting information in the memory 23. A plurality of sets of single event counting information generated and accumulated by the identification function 241 are Compton camera raw data.

The image generation function 242 reconstructs the PET image based on the Compton camera raw data (single event counting information). For example, the image generation function 242 calculates the energy lost due to Compton scattering based on the energy of the scattered gamma ray and the energy (511 keV) of the annihilation gamma ray for each set of single event counting information. The image generation function 242 calculates the scattering angle for each set of single event counting information and determines each cone based on the scattering angle, the Compton scattering point, and the detection position of the scattered gamma ray. The image generation function 242 superimposes a plurality of cones to identify the spatial distribution of the gamma-ray source and reconstructs the PET image that depicts the image of the gamma-ray source.

When the scatterer detector 1 is a TPC, the identification function 241 reads the first counting information and the second counting information including the information on the track of the recoil electron from the memory 23 and generates the single event counting information having the first counting information and the second counting information associated with each other based on the position of the Compton scattering point, the incident position of the scattered gamma ray, and each detection time. Specifically, the identification function 241 generates the single event counting information in which the Compton scattering point, the information on the three-dimensional position of the track of the recoil electron, the incident position of the scattered gamma ray, and the energy of the scattered gamma ray are associated with each other. The position of the Compton scattering point may be obtained from the information on the three-dimensional position of the track of the recoil electron.

FIG. 4 is a diagram illustrating an example of generation of the single event counting information by the identification function 241 according to the first embodiment. For example, the identification function 241 uses the position of the Compton scattering point, the detection position of the scattered gamma ray, and each detection time to associate the information on the Compton scattering point and the track of the recoil electron included in the first counting information with the energy and the detection position of the scattered gamma ray included in the second counting information, as illustrated in FIG. 4, so as to identify the single event. The identification function 241 stores, in the memory 23, the single event counting information including the information on the Compton scattering point and the track of the recoil electron included in the first counting information and the energy and the detection position of the scattered gamma ray included in the second counting information.

The identification function 241 uses the first counting information including the information on the track of the recoil electron generated by the first acquisition circuitry 101 and the second counting information generated by the second acquisition circuitry 102 to sequentially generate the single event counting information with regard to the annihilation gamma ray emitted from the subject P and stores the single event counting information in the memory 23. A plurality of sets of single event counting information generated and accumulated by the identification function 241 are electron-track detection Compton camera raw data.

The image generation function 242 reconstructs the PET image based on the electron-track detection Compton camera raw data (single event counting information). For example, the image generation function 242 calculates the energy lost due to Compton scattering based on the energy of the scattered gamma ray and the energy (511 keV) of the annihilation gamma ray for each set of single event counting information. The image generation function 242 calculates the scattering angle for each set of single event counting information. The image generation function 242 specifies the incident direction of the incident gamma ray based on the scattering angle, the Compton scattering point, the detection position of the scattered gamma ray, and the information on the three-dimensional position of the track of the recoil electron. Further, the image generation function 242 superimposes the incident directions of the incident gamma rays for each set of single event counting information to specify the spatial distribution of the gamma-ray source and reconstructs the PET image that depicts the image of the gamma-ray source.

In the case described above, the energy lost due to Compton scattering is calculated from the energy of the scattered gamma ray and the energy (511 keV) of the annihilation gamma ray. However, the embodiment is not limited thereto, and for example, when the energy of the recoil electron is acquired, the energy of the recoil electron may be the energy lost due to Compton scattering.

As described above, the identification function 241 identifies the single event. Here, the identification function 241 may also identify a coincidence counting event of gamma rays that are incident on the PET detector 2 without being scattered by the scatterer detector 1. In such a case, the identification function 241 first determines whether the gamma ray detected by the PET detector 2 is incident on the PET detector 2 without being scattered by the scatterer detector 1.

For example, the identification function 241 determines whether the detected gamma ray is incident on the PET detector 2 without being scattered by the scatterer detector 1 based on the energy of the gamma ray included in the second counting information. As an example, the identification function 241 determines whether the gamma ray is incident on the PET detector 2 without being scattered by the scatterer detector 1 based on whether the energy of the gamma ray included in the second counting information falls within a preset range. The range used for determination may be optionally set and is set based on, for example, a change in the energy due to scattering.

The identification function 241 identifies a coincidence counting event of gamma rays that are incident on the PET detector 2 without being scattered by the scatterer detector 1. For example, the identification function 241 retrieves, in the second counting information corresponding to the gamma rays that are determined to have been incident on the PET detector 2 without being scattered by the scatterer detector 1, a combination of gamma rays whose detection times fall within a time window width of a certain time period and whose energy values both fall within a certain energy window width.

The identification function 241 determines that the second counting information on the retrieved combination is the information obtained by coincidentally counting the two annihilation photons and generates the coincidence counting information. The line connecting the two detection positions where two annihilation photons are coincidentally counted is called LOR (Line of Response).

The image generation function 242 reconstructs the PET image based on the single event counting information and the coincidence counting information. Specifically, the image generation function 242 generates the PET image based on the Compton camera raw data or the electron-track detection Compton camera raw data and the coincidence counting information.

As described above, the identification function 241 identifies the coincidence counting event of the gamma rays that are incident on the PET detector 2 without being scattered by the scatterer detector 1. Here, the identification function 241 may also identify the coincidence counting event of the gamma ray that is incident on the PET detector 2 without being scattered by the scatterer detector 1 and the scattered gamma ray that is scattered by the scatterer detector 1. In such a case, the identification function 241 retrieves, in the single event counting information and the second counting information, the combination of gamma rays whose detection times fall within the time window width of a certain time period.

Here, in the case of the coincidence counting event of the gamma ray that is incident on the PET detector 2 without being scattered by the scatterer detector 1 and the scattered gamma ray scattered by the scatterer detector 1, the LOR is the straight line connecting the Compton scattering point and the detection position of the gamma ray that is incident on the PET detector 2 without being scattered by the scatterer detector 1.

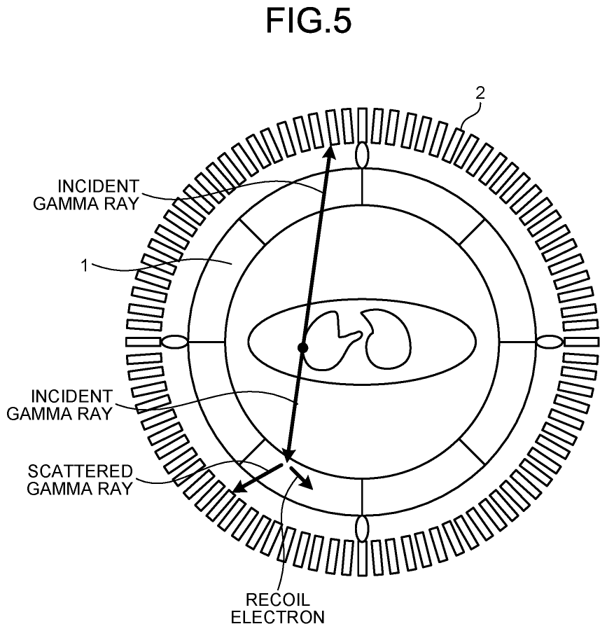

FIG. 5 is a diagram illustrating an example of the LOR according to the first embodiment. In the case of the coincidence counting event of the gamma ray detected by the PET detector 2 without Compton scattering and the scattered gamma ray, as illustrated in for example FIG. 5, the LOR is the line segment connecting the detection position of the incident gamma ray that is incident on the PET detector 2 without being scattered by the scatterer detector 1 and the Compton scattering point of the incident gamma ray having undergone Compton scattering by the scatterer detector 1. The image generation function 242 generates the PET image using the LOR illustrated in FIG. 5.

As described above, the PET apparatus 100 according to the first embodiment includes the scatterer detector 1 to execute a Compton camera only mode for generating the PET image based on the Compton camera raw data, an electron-track detection Compton camera only mode for generating the PET image based on the electron-track detection Compton camera row data, a Compton camera & PET simultaneous mode for generating the PET image based on the Compton camera raw data and the coincidence counting information, and an electron-track detection Compton camera & PET simultaneous mode for generating the PET image based on the electron-track detection Compton camera raw data and the coincidence counting information.

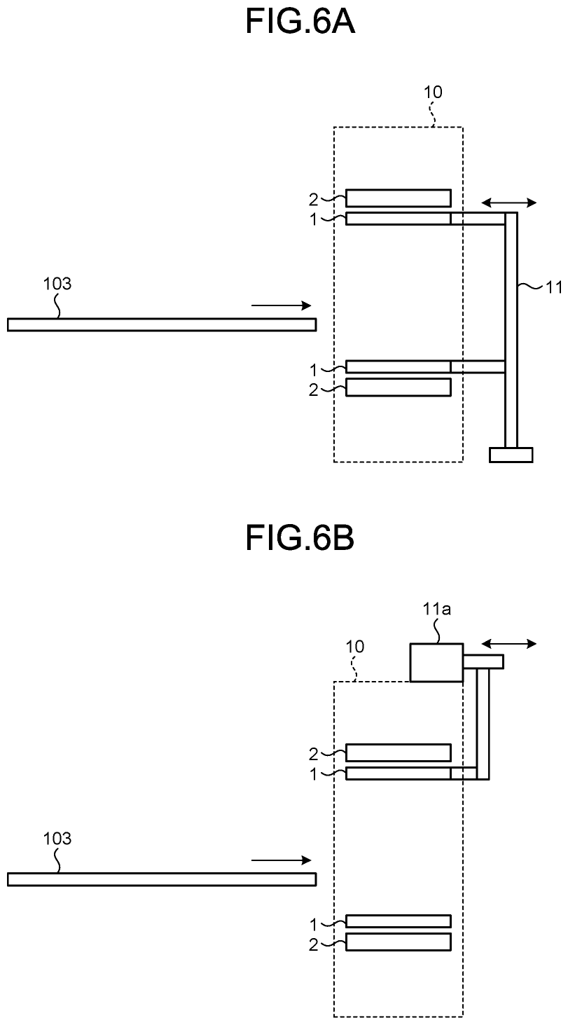

Here, the scatterer detector 1 in the PET apparatus 100 may be detachably mounted. FIGS. 6A and 6B are diagrams illustrating an installation example of the scatterer detector 1 according to the first embodiment. For example, as illustrated in FIG. 6A, the scatterer detector 1 is supported by a support unit 11 that is propelled by itself in the direction indicated by an arrow. The support unit 11 moves toward the gantry 10 from the side opposite to the top plate 103 with the gantry 10 interposed therebetween so that the scatterer detector 1 is inserted into the imaging port and is located inside the PET detector 2. When the support unit 11 moves in a direction away from the gantry 10, the scatterer detector 1 is pulled out from the imaging port, and only the PET detector 2 is located in the gantry 10 in this state.

For example, the scatterer detector 1 is held by an arm mechanism 11a installed in an upper portion of the gantry 10, as illustrated in FIG. 6B. The arm mechanism 11a is configured to be expandable and contractible in a direction indicated by an arrow in FIG. 6B. When the arm mechanism 11a contracts, the scatterer detector 1 is inserted into the imaging port and is located inside the PET detector 2. When the arm mechanism 11a extends, the scatterer detector 1 is pulled out from the imaging port, and only the PET detector 2 is located in the gantry 10 in this state.

As described above, the scatterer detector 1 may be detachably mounted on the gantry 10. When the scatterer detector 1 is removed from the gantry 10, the PET apparatus 100 is in the PET only mode for generating the PET image based on the coincidence counting information.

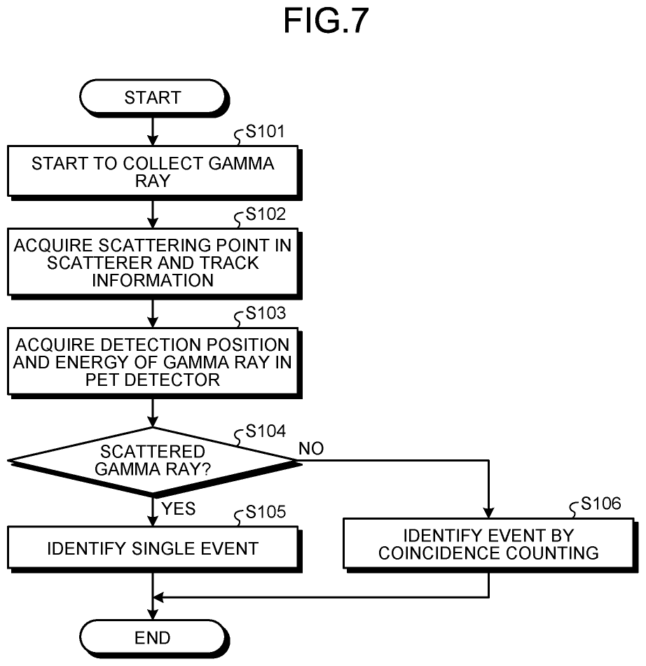

An example of processing by the PET apparatus 100 is described below with reference to FIG. 7. FIG. 7 is a flowchart illustrating a processing procedure by the PET apparatus 100 according to the first embodiment. FIG. 7 illustrates processing when the electron-track detection Compton camera & PET simultaneous mode is executed.

Here, Step S101 in FIG. 7 is performed when, for example, the processing circuitry 24 reads and executes a program corresponding to the system control function 243 from the memory 23. Step S102 is performed by the first acquisition circuitry 101. Step S103 is performed by the second acquisition circuitry 102. Steps S104 to S106 are performed when, for example, the processing circuitry 24 reads and executes a program corresponding to the identification function 241 from the memory 23.

In the PET apparatus 100 according to the first embodiment, as illustrated in FIG. 7, the processing circuitry 24 starts to collect a gamma ray (Step S101), and the first acquisition circuitry 101 acquires the Compton scattering point in the scatterer and the track information on the recoil electron (Step S102).

The second acquisition circuitry 102 acquires the detection position and the energy of the gamma ray in the PET detector 2 (Step S103). Subsequently, the processing circuitry 24 determines whether the gamma ray detected by the PET detector 2 is a scattered gamma ray (Step S104).

Here, when the gamma ray detected by the PET detector 2 is a scattered gamma ray (Yes at Step S104), the processing circuitry 24 identifies a single event (Step S105). Conversely, when the gamma ray detected by the PET detector 2 is not a scattered gamma ray (No at Step S104), the processing circuitry 24 identifies a coincidence counting event (Step S106).

As described above, according to the first embodiment, the scatterer detector 1 is provided inside the PET detector. The identification function 241 detects the gamma ray scattered by the scatterer detector 1 with the PET detector 2 to identify the single event. Therefore, the PET apparatus 100 according to the first embodiment makes it possible to easily implement the Compton camera in the nuclear medicine diagnostic apparatus.

According to the first embodiment, the scatterer detector 1 is annularly provided inside the PET detector 2. Therefore, the PET apparatus 100 according to the first embodiment makes it possible to implement the Compton camera that may obtain a sufficient image quality applicable to medical diagnosis.

According to the first embodiment, the scatterer detector 1 is detachably mounted inside the PET detector 2. Therefore, the PET apparatus 100 according to the first embodiment makes it possible to easily switch between the Compton camera mode and the PET mode.

According to the first embodiment, the identification function 241 detects the pair of annihilation gamma rays with the PET detector 2 to further identify the coincidence counting event. Therefore, the PET apparatus 100 according to the first embodiment may detect the pair of annihilation gamma rays while in the Compton camera mode and may improve the image quality.

According to the first embodiment, the identification function 241 further identifies the coincidence counting event based on the timing at which the PET detector 2 detects the gamma ray scattered by the scatterer detector 1 and the timing at which the PET detector 2 detects the annihilation gamma ray. Therefore, the PET apparatus 100 according to the first embodiment may conduct coincidence counting of the scattered gamma ray and the annihilation gamma ray while in the Compton camera mode and may further improve the image quality.

According to the first embodiment, the scatterer detector 1 is a time projection chamber. Therefore, the PET apparatus 100 according to the first embodiment may execute the electron-track detection Compton camera mode.

### Second Embodiment

In the first embodiment described above, the PET apparatus 100 is described as an example of a nuclear medicine diagnostic apparatus. In a second embodiment, a SPECT (single photon emission computed tomography) apparatus is described as an example of a nuclear medicine diagnostic apparatus.

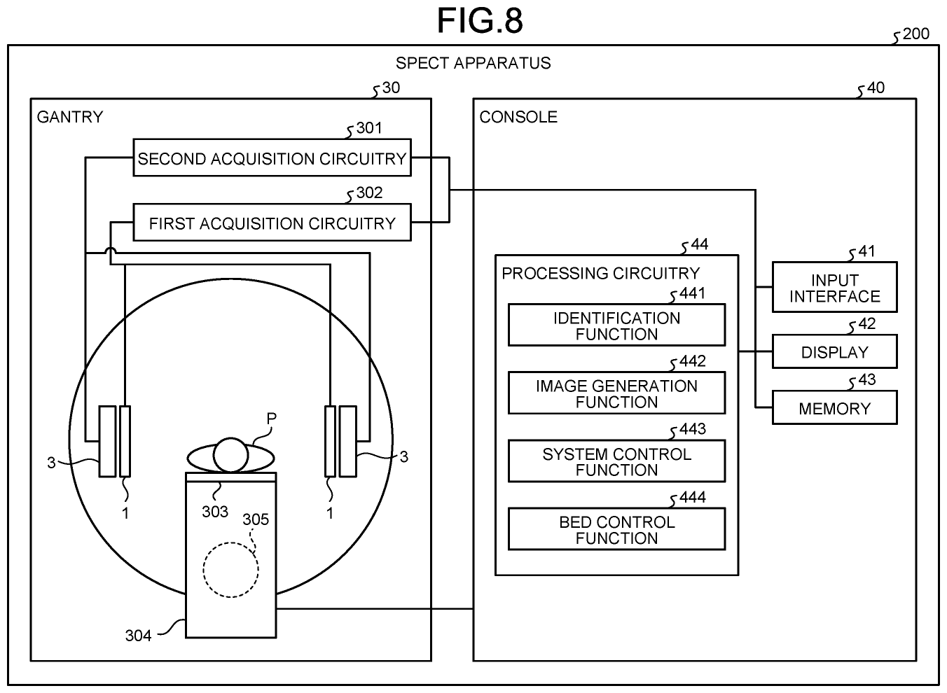

FIG. 8 is a diagram illustrating an example of a configuration of a SPECT apparatus 200 according to the second embodiment. As illustrated in FIG. 8, the SPECT apparatus 200 according to the second embodiment includes a gantry 30 and a console 40.

The gantry 30 includes the scatterer detector 1, a gamma ray detector 3, first acquisition circuitry 302, second acquisition circuitry 301, a top plate 303, a bed 304, and a bed drive unit 305 to detect gamma rays emitted from a living tissue that takes up a single-photon emitting radionuclide administered to the subject P and thus generates counting information for reconstructing the SPECT image.

The scatterer detector 1 causes Compton scattering of the gamma ray emitted from the subject P and detects the Compton scattering point. For example, as illustrated in FIG. 8, the scatterer detector 1 is provided inside the gamma ray detector 3. Specifically, the scatterer detector 1 is provided between the subject P and the gamma ray detector 3. The scatterer detector 1 is detachably mounted inside the gamma ray detector 3. For example, the scatterer detector 1 is replaced with a collimator to be provided inside the gamma ray detector 3. Here, the scatterer detector 1 may have the same configuration as that in the first embodiment described above. That is, the scatterer detector 1 may also be configured by a time projection chamber.

As illustrated in FIG. 8, the gamma ray detector 3 is provided outside the scatterer detector 1 to detect the gamma ray emitted from the living tissue of the subject P. Specifically, the gamma ray detector 3 detects the scattered gamma ray having undergone Compton scattering by the scatterer detector 1. When the scatterer detector 1 is replaced with the collimator, the gamma ray detector 3 detects the gamma ray emitted from the living tissue of the subject P.

For example, the gamma ray detector 3 is a detector of photon counting method or anger type, including a scintillator, a photodetection element, and a light guide.

The scintillator converts incident annihilation gamma rays, which are emitted from the positrons in the subject P, into scintillation lights (scintillation photons, optical photons) and outputs the scintillation lights. The scintillator is formed of scintillator crystals suitable for energy measurement, such as LaBr3 (lanthanum bromide), LYSO (lutetium yttrium oxyorthosilicate), LSO (lutetium oxyorthosilicate), or LGSO (lutetium gadolinium oxyorthosilicate), or BGO, and is arranged in for example two dimensions.

For example, SiPM (Silicon photomultiplier) or a photomultiplier tube is used as a photodetection element. The photomultiplier tube includes a photocathode that receives a scintillation light and generates a photoelectron, a multi-stage dynode that gives an electric field to accelerate the generated photoelectron, and an anode that is an electron outflow port to multiply the scintillation light output from the scintillator and convert it into an electrical signal.

The light guide is formed of a plastic material, or the like, having desired light transmission to transmit the scintillation light output from the scintillator to the photodetection element such as SiPM or photomultiplier tube.

The gamma ray detector 3 is an example of a gamma ray detector. Although FIG. 8 illustrates a case where the two scatterer detectors 1 and the two gamma ray detectors 3 are provided, the embodiment is not limited thereto, and the three or more scatterer detectors 1 and the three or more gamma ray detectors 3 may be provided.

The first acquisition circuitry 302 generates the first counting information from the output signal of the scatterer detector 1 and stores the generated first counting information in a memory 43 of the console 40 as in the first embodiment described above.

The second acquisition circuitry 301 generates the second counting information from the output signal of the gamma ray detector 3 and stores the generated second counting information in the memory 43 of the console 40 as in the first embodiment described above.

The first acquisition circuitry 301 and the second acquisition circuitry 302 are implemented by, for example, a CPU (central processing unit), a GPU (graphical processing unit), or a circuit such as an application specific integrated circuit (ASIC), a programmable logic device (e.g., simple programmable logic device (SPLD), complex programmable logic device (CPLD), and a field programmable gate array (FPGA)).

The top plate 303 is a bed on which the subject P is placed and is provided on the bed 304. The bed drive unit 305 moves the top plate 303 under the control of a bed control function 444 of processing circuitry 44. For example, the bed drive unit 305 moves the top plate 303 to move the subject P into an imaging port of the gantry 30.

The console 40 receives an operation on the SPECT apparatus 200 by the operator, controls capturing of a SPECT image, and reconstructs the SPECT image using the counting information collected by the gantry 30. As illustrated in FIG. 8, the console 40 includes an input interface 41, a display 42, the memory 43, and the processing circuitry 44. The units included in the console 40 are coupled to each other via a bus.

The input interface 41 is implemented by, for making various settings, and the like, a trackball, a switch button, a mouse, a keyboard, a touch pad for performing an input operation by touching an operation surface, a touch monitor having a display screen and a touch pad integrated therein, a non-contact input circuit using an optical sensor, a voice input circuit, etc. The input interface 41 is coupled to the processing circuitry 44 to convert the input operation received from the operator into an electric signal and output the electric signal to the processing circuitry 44. In this description, the input interface 41 is not limited to the one including a physical operating part such as a mouse and a keyboard. Examples of the input interface include an electric signal processing circuit that receives an electric signal corresponding to an input operation from an external input device, which is provided separately from the apparatus, and outputs the electric signal to the processing circuitry 44.

The display 42 is coupled to the processing circuitry 44 to display various types of information and various types of image data output from the processing circuitry 44. For example, the display 42 is implemented by a liquid crystal display, a CRT (cathode ray tube) display, an organic EL display, a plasma display, or a touch panel. According to the present embodiment, for example, the display 42 presents a SPECT image or presents a GUI (graphical user interface), or the like, for receiving various instructions and various settings from the operator.

The memory 43 stores various types of data used in the SPECT apparatus 200. The memory 43 is implemented by, for example, a semiconductor memory device such as a RAM (random access memory) or a flash memory, a hard disk, or an optical disk. The memory 43 stores the first counting information acquired by the first acquisition circuitry 302, the second counting information acquired by the second acquisition circuitry 301, the single event counting information in which the identification number for identifying the single event is associated with the first counting information and the second counting information, the reconstructed SPECT image, etc.

As illustrated in FIG. 8, the processing circuitry 44 executes an identification function 441, an image generation function 442, a system control function 443, and the bed control function 444. Here, according to the embodiment, each of the processing functions performed by the identification function 441, the image generation function 442, the system control function 443, and the bed control function 444 is stored in the memory 43 in the form of a program executable by a computer. The processing circuitry 44 is a processor that reads and executes a program from the memory 43 to perform the function corresponding to each program. In other words, the processing circuitry 44 having read each program has each function illustrated in the processing circuitry 44 of FIG. 8. In the description of FIG. 8, it is assumed that the single processing circuitry 44 performs the identification function 441, the image generation function 442, the system control function 443, and the bed control function 444, but a plurality of independent processors may be combined to form the processing circuitry 44, and each of the processors may execute a program to perform the function. In other words, each of the above-described functions may be configured as a program, and the single processing circuitry 44 may execute each program. As another example, a specific function may be installed in a dedicated and independent program execution circuit.

The identification function 441 detects the gamma ray scattered by the scatterer with the gamma ray detector 3 to identify a single event. Specifically, in the same manner as the identification function 241 described in the first embodiment, the identification function 441 generates the single event counting information based on the first counting information and the second counting information and stores the generated single event counting information in the memory 43. The identification function 441 is an example of an identification unit.

The image generation function 442 reconstructs the SPECT image using the counting information. Specifically, the image generation function 442 reads the single event counting information stored in the memory 43 and uses the read single event counting information to reconstruct the SPECT image. Specifically, the image generation function 442 generates the SPECT image based on the Compton camera raw data and the SPECT image based on the electron-track detection Compton camera raw data. When the collimator is attached instead of the scatterer detector 1, the image generation function 442 reads the second counting information stored in the memory 43 and uses the read second counting information to reconstruct the SPECT image. The image generation function 442 stores the reconstructed SPETC image in the memory 43.

The system control function 443 controls the gantry 30 and the console 40 to perform the overall control on the SPECT apparatus 200. For example, the system control function 443 controls imaging in the SPECT apparatus 200.

The bed control function 444 controls the bed drive unit 305 to control the position of the top plate 303.

As described above, the SPECT apparatus 200 according to the second embodiment includes the scatterer detector 1 to execute a Compton camera only mode for generating the SPECT image based on the Compton camera raw data, an electron-track detection Compton camera only mode for generating the SPECT image based on the electron-track detection Compton camera raw data, and a SPECT only mode when the collimator is mounted.

As described above, according to the second embodiment, the scatterer detector 1 is detachably mounted inside the gamma ray detector 3. The identification function 441 detects the gamma ray scattered by the scatterer detector 1 with the gamma ray detector 3 to identify the single event. Therefore, the SPECT apparatus 200 according to the second embodiment makes it possible to easily implement the Compton camera in the nuclear medicine diagnostic apparatus.

According to the second embodiment, the scatterer detector 1 is replaced with the collimator in the gamma ray detector 3 to be provided inside the gamma ray detector 3. Therefore, the SPECT apparatus 200 according to the second embodiment makes it possible to easily switch between the Compton camera mode and the SPECT mode.

For example, when the incident direction of gamma rays is specified by the collimator, only approximately 1/1000 of the gamma rays emitted from the subject is detectable. However, by executing the Compton camera mode, it is possible to increase the amount of detected gamma rays that are emitted from the subject.

According to the second embodiment, the scatterer detector 1 is a time projection chamber. Therefore, the SPECT apparatus 200 according to the second embodiment may execute the electron-track detection Compton camera mode.

### Other Embodiments

Although the first embodiment and the second embodiment are described above, various different embodiments may be implemented in addition to the first embodiment and the second embodiment described above.

In the description according to the above-described embodiment, the PET apparatus 100 uses one radionuclide. However, the embodiment is not limited thereto, and imaging using a plurality of radionuclides may be executed. For example, the PET apparatus 100 may generate the image corresponding to each energy with regard to the subject P having administered therein a medical agent using a plurality of radionuclides that emit gamma rays having significantly different original energies.

In such a case, for example, an energy window for specifying a gamma ray of each energy is previously set, and the identification function 241 uses the energy window to separate the counting information and uses the separated counting information to generate the single event counting information corresponding to each energy.

The image generation function 242 uses the single event counting information corresponding to each energy to reconstruct the image corresponding to each energy.

The term “processor” used in the above description refers to, for example, a CPU (central processing unit), a GPU (graphical processing unit), or a circuit such as an application specific integrated circuit (ASIC), a programmable logic device (e.g., simple programmable logic device (SPLD), complex programmable logic device (CPLD), or field programmable gate array (FPGA)). Here, a configuration may be such that, instead of storing the program in the memory, the program is directly installed in a circuit of the processor. In this case, the processor reads and executes the program installed in the circuit to perform the function. Each processor according to the present embodiment is not always configured as one circuit on a per-processor basis, but a plurality of independent circuits may be combined to configure one processor to perform the function.

Here, the program executed by the processor is provided by being previously installed in a ROM (read only memory), a storage unit, or the like. The program may be provided by being recoded in a computer-readable storage medium, such as a CD (compact disk)-ROM, FD (flexible disk), CD-R (recordable), or DVD (digital versatile disk) in the form of file installable or executable on these devices. The program may be provided or distributed by being stored on a computer connected to a network such as the Internet and downloaded via the network. For example, the program includes modules including each functional unit. As the actual hardware, the CPU reads and executes a program from a storage medium such as a ROM so that each module is loaded on a primary storage device and generated on the primary storage device.

According to at least one of the embodiments described above, the Compton camera may be easily implemented in the nuclear medicine diagnostic apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nuclear medicine diagnostic apparatus comprising:
a positron emission tomography (PET) detector;
a scatterer provided inside the PET detector; and
processing circuitry configured to detect a gamma ray scattered by the scatterer with the PET detector to identify a single event.

2. The nuclear medicine diagnostic apparatus according to claim 1, wherein the scatterer is annularly provided inside the PET detector.

3. The nuclear medicine diagnostic apparatus according to claim 1, wherein the scatterer is detachably mounted inside the PET detector.

4. The nuclear medicine diagnostic apparatus according to claim 1, wherein the processing circuitry is configured to detect a pair of annihilation gamma rays with the PET detector to further identify a coincidence counting event.

5. The nuclear medicine diagnostic apparatus according to claim 1, wherein the processing circuitry is further configured to identify a coincidence counting event based on timing at which the PET detector detects the gamma ray scattered by the scatterer and timing at which the PET detector detects an annihilation gamma ray.

6. The nuclear medicine diagnostic apparatus according to claim 1, wherein the scatterer is a time projection chamber.

7. A nuclear medicine diagnostic apparatus comprising:
a gamma ray detector;
a scatterer that is detachably mounted inside the gamma ray detector; and processing circuitry configured to detect a gamma ray scattered by the scatterer with the gamma ray detector to identify a single event.

8. The nuclear medicine diagnostic apparatus according to claim 7, wherein the scatterer is replaced with a collimator in the gamma ray detector to be provided inside the gamma ray detector.

9. The nuclear medicine diagnostic apparatus according to claim 7, wherein the scatterer is a time projection chamber.
